(19) United States
(12) Patent Application Publication
(10) Pub. No.: US 2010/0312118 A1
(43) Pub. Date: Dec. 9, 2010

(54) Systems and Methods for Perfusion Enhanced Diagnostic Imaging

(76) Inventor: Michael J. Horzewski, San Jose, CA (US)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(21) Appl. No.: 12/782,196

(22) Filed: May 18, 2010

Related U.S. Application Data
(60) Provisional application No. 61/183,890, filed on Jun. 3, 2009.

(51) Int. Cl. A61B 8/14

(52) U.S. Cl. 600/458

(57) ABSTRACT

Systems and methods for enhancing diagnostic imaging by the means of increasing tissue perfusion and/or vasodilation, and thus, increasing imaging agent perfusion distribution and/or imaging agent uptake by tissue. The desired effects can be achieved by exposing the area of interest to ultrasound either before or after image acquisition, or in between multiple image acquisitions.

### RELATED INFORMATION

This application claims the benefit of U.S. provisional application No. 61/183,890, filed Jun. 3, 2009, which application is incorporated herein by reference.

### FIELD OF THE INVENTION

This invention relates to systems and methods for enhancing diagnostic imaging by the means of increasing tissue perfusion and/or vasodilation.

### BACKGROUND OF THE INVENTION

In diagnostic imaging, depending on the imaging technology, imaging agents are used to enhance the image contrast, or to facilitate the visualization of areas of interest in the image. Examples of imaging agents are micro bubbles in ultrasound imaging, contrast agents in magnetic resonance imaging (MRI), and radionuclides in nuclear imaging (SPECT, single photon emission computed tomography, or PET, positron emission tomography).

Vasodilation is a term that describes the increase in the internal diameter of a blood vessel that results from relaxation of smooth muscle within the wall of the vessel. Vasodilation causes an increase in blood flow, as well as a corresponding decrease in systemic vascular resistance (i.e., reduced blood pressure). Tissue perfusion is a term that generally describes fluid flow through the lymphatic system, or blood vessels into an organ or tissue.

The effects of ultrasound energy upon enhanced perfusion and/or vasodilation have been observed and widely reported in scientific literature. The combination of ultrasound exposure and diagnostic imaging has the potential of increasing imaging agent perfusion distribution, and/or imaging agent uptake by tissue, and therefore, enhancement of diagnostic image quality. Enhanced image quality, in turn, results in a quicker and more accurate choice of treatment on patients than conventional diagnostic imaging without the effects of ultrasound. Depending on the diagnostic imaging technology, the ultrasound exposure can occur either before or after image acquisition, or in between multiple image acquisitions.

### BRIEF SUMMARY OF THE INVENTION

The invention provides systems and methods for enhancing diagnostic imaging by the means of increasing tissue perfusion and/or vasodilation, and thus, increasing imaging agent perfusion distribution and/or imaging agent uptake by tissue. The desired effects can be achieved by exposing the area of interest to ultrasound either before or after image acquisition, or in between multiple image acquisitions.

Other features and advantages of the inventions are set forth in the following specification and attached drawings.

### BRIEF DESCRIPTION OF THE DRAWINGS

FIG. 1 is a perspective view of a system for transcutaneously applying ultrasound energy to affect vasodilation and/or increased blood perfusion.

FIG. 2 is an enlarged exploded perspective view of an ultrasound energy applicator that forms a part of the system shown in FIG. 1.

FIG. 3 is an enlarged assembled perspective view of the ultrasound energy applicator shown in FIG. 2.

FIG. 4 is a side section view of the acoustic contact area of the ultrasound energy applicator shown in FIG. 2.

FIG. 5 is a view of the applicator shown in FIG. 2 held by a stabilization assembly in a secure position overlaying the sternum of a patient, to transcutaneously direct ultrasonic energy, e.g., toward the vasculature of the heart.

FIG. 6 is a side elevation view, with portions broken away and in section, of an acoustic stack that can be incorporated into the applicator shown in FIG. 2.

FIG. 7 is a side elevation view, with portions broken away and in section, of an acoustic stack that can be incorporated into the applicator shown in FIG. 2.

FIGS. 8a to 8c graphically depicts the technical features of a frequency tuning function that the system shown in FIG. 1 can incorporate.

FIG. 9 graphically depicts the technical features of a power ramping function that the system shown in FIG. 1 can incorporate.

FIG. 10 is a schematic view of a controller that the system shown in FIG. 1 can incorporate, which includes a frequency selection and tuning function, a power ramping function, and an output power control function.

FIG. 11 is a diagrammatic view of a use register chip that forms a part of the use monitoring function shown in FIG. 10.

FIG. 12 is a diagrammatic flow chart showing the technical features of the use monitoring function shown in FIG. 10.

FIG. 13 shows an example about the use of the system shown in FIG. 1 when used in conjunction with PET (positron emission tomography) imaging.

FIG. 14 shows the distribution of the perfusion tracer (rubidium-82) in PET images prior and post ultrasound treatment using the system shown in FIG. 1.

FIG. 15 shows the distribution of the perfusion tracer (rubidium-82) in an alternate representation of PET images prior and post ultrasound treatment using the system shown in FIG. 1.

The invention may be embodied in several forms without departing from its spirit or essential characteristics. The scope of the invention is defined in the appended claims, rather than in the specific description preceding them. All embodiments that fall within the meaning and range of equivalency of the claims are therefore intended to be embraced by the claims.

### DESCRIPTION OF THE PREFERRED EMBODIMENTS

A system 10 will be described in connection with the diagnostic image enhancement by the means of providing increased vasodilation and/or increase blood perfusion by the transcutaneous application of ultrasound energy. As used herein, the term “ultrasound energy” means vibrational energy in a range of frequencies greater than about 20 kHz.

The ultrasound energy is desirably indicated, e.g., for the enhancement of any diagnostic imaging modality; and/or before, during, or after image acquisition; and/or in between multiple image acquisitions; and/or before, during, or after imaging agent injection or administration; and/or in between imaging agent injection or administration, or multiple injections or administrations. The system 10 has application for use in diverse regions of the body, e.g., in the thoracic cavity, the arms, the legs, or the head.

I. System for Providing Noninvasive Vasodilation and/or Blood Perfusion Using Ultrasound Energy

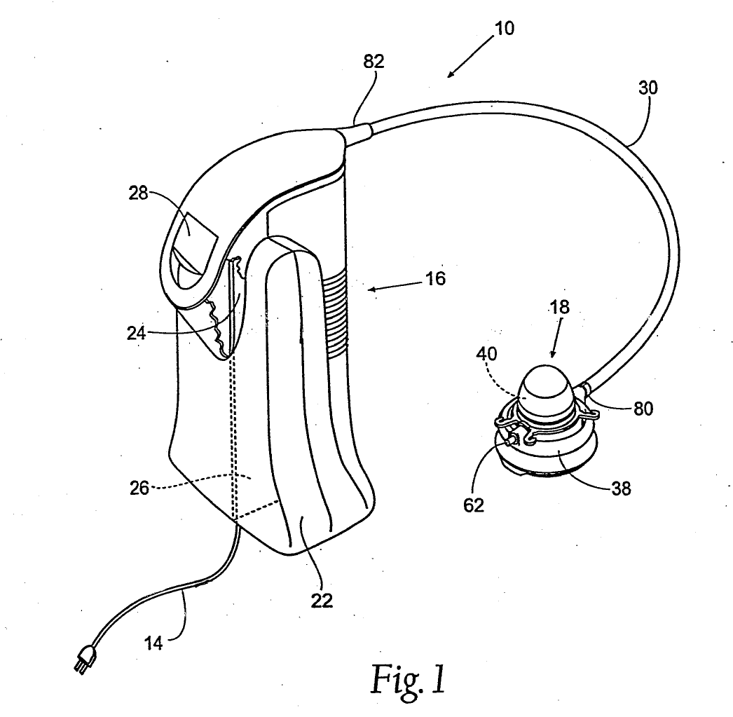

FIG. 1 schematically shows a compact, portable ultrasound system 10 that makes it possible to apply ultrasound energy on a person who needs or who is likely to need vasodilation and/or an increase in the flow rate or perfusion of circulating blood.

The system 10 includes durable and disposable equipment and materials necessary to apply ultrasound on the person at a designated treatment location. In use, the system 10 affects increased vasoldilation and/or blood perfusion by transcutaneously applying ultrasound energy.

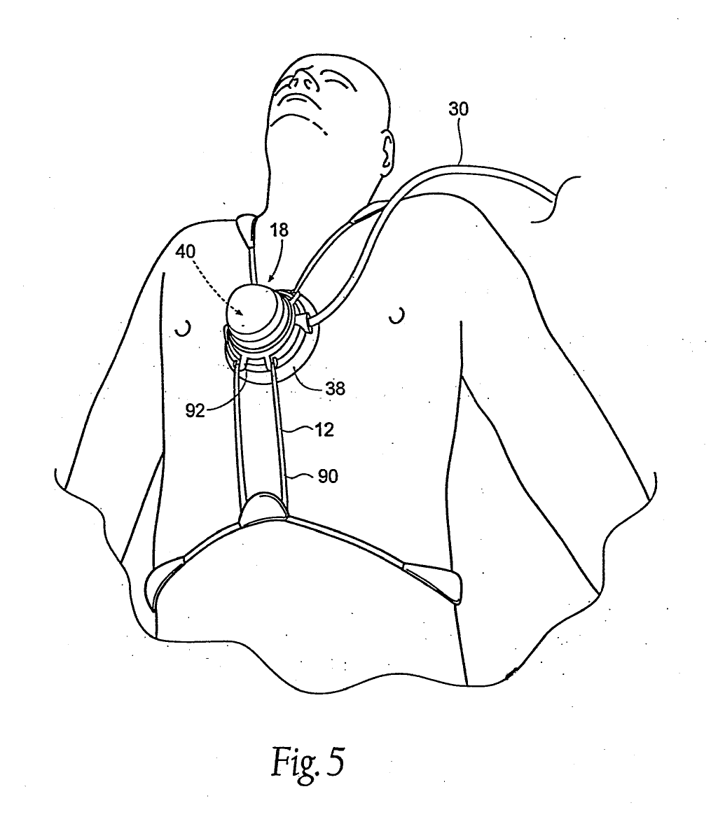

As FIG. 1 shows, the system 10 includes at the treatment location an ultrasound energy generating machine 16. The system 10 also includes at the treatment location at least one ultrasound energy applicator 18, which is coupled to the machine 16 during use. As FIG. 5 shows, the system 10 also includes an assembly 12 for use with the applicator 18 to stabilize the position of the applicator 18 on a patient for hands-free use. In the illustrated embodiment (see FIG. 5), the applicator 18 is secured against movement on a person's thorax, overlaying the sternum, to direct ultrasonic energy toward the vasculature of the heart. It should be appreciated that the applicator can be sized and configured for secure placement on other regions of the body, such as the arms, legs, or head.

The location where ultrasound exposure occurs can vary. It can be a traditional clinical setting, where support and assistance by one or more medically trained care givers are immediately available to the person, such as inside a hospital, e.g., in an emergency room, catheter lab, operating room, or critical care unit. However, due to the purposeful design of the system 10, the location need not be confined to a traditional clinical setting. The location can comprise a mobile setting, such as an ambulance, helicopter, airplane, or like vehicle used to convey the person to a hospital or another clinical treatment center. The location can even comprise an everyday, public setting, such as on a cruise ship, or at a sports stadium or airport, or a private setting, such as in a person's home, where the effects of vasoconstriction and/or low blood perfusion can arise.

By purposeful design of durable and disposable equipment, the system 10 can make it possible to initiate treatment of vasoconstriction and/or a reduced blood perfusion incident in a non-clinical, even mobile location, outside a traditional medical setting. The system thereby makes effective use of the critical time period before the person enters a hospital or another traditional medical construction treatment center.

The features, construction and operation of the system 10 will be described in greater detail later, but first some examples will be discussed.

II. Use with an Imaging Agent

The system 10 in FIG. 1 can further include a delivery system for introducing an imaging agent in conjunction with the use of the applicator 18 and machine 16. In this arrangement, the effect of vasodilation and/or increased tissue perfusion caused by the application of ultrasound energy can increase the imaging agent perfusion distribution, and/or imaging agent uptake by tissue, and therefore enhance the diagnostic image quality. Enhanced image quality, in turn, results in a quicker and more accurate choice of treatment on patients than conventional diagnostic imaging without the effects of ultrasound.

Preferably, the imaging agent is introduced into the area of interest, prior to, in conjunction with, or after the application of ultrasound. The imaging agent can be administered into the body by intravenous injection in liquid or aggregate form, or ingestion while combined with food, or inhalation as a gas or aerosol. The interaction between the applied ultrasound and the imaging agent is observed to enhance the image contrast by the means of increasing tissue perfusion and/or vasodilation, and thus, increasing imaging agent perfusion distribution and/or imaging agent uptake by tissue.

The type of imaging agent used can vary. The imaging agent can be a radionuclide, such as thallium-201 (²⁰¹T1) or technetium-99m (^{99m}Tc); or nitrogen-13 ammonia (13N-ammonia) or rubidium-82 (⁸²Rb). Alternatively, the imaging agent can comprise micro bubbles, or contrast agents, such as barium or iodine; or gadolinium or manganese.

The area of interest can vary, according to the region of the body. For example, in the thoracic cavity, the use of ultrasonic energy can help diagnose “hibernating myocardium”, or ischemic heart disease.

Depending on the imaging modality, it may be possible to reduce the typical dose of imaging agent when ultrasonic energy is also applied. The ability to reduce the dosage of imaging agent, when ultrasound is also applied, can lead to additional benefits, such as reduced exposure to ionizing radiation, or an increased patient population eligible for the diagnosis.

### Examples

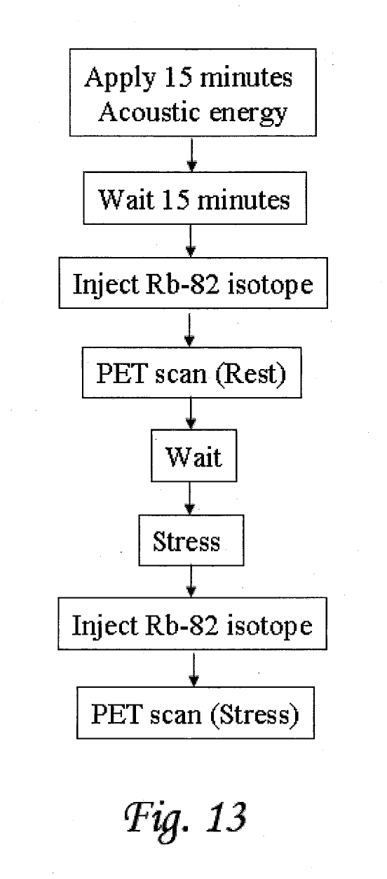

FIG. 13 shows an example about the implementation of the subject invention to nuclear imaging of the heart, specifically PET (positron emission tomography) imaging for the diagnosis of myocardial blood flow (MBF). In this imaging protocol, as shown in FIG. 13, the ultrasound treatment using the system shown in FIG. 1 occurs in between two successive sets of PET imaging sessions. The ultrasound treatment time is 15 minutes, followed by a 15-minute response time to treatment. Each PET imaging session comprises two scans, a transmission scan (Rest Tx Scan) and emission scan (Emission Scan 2D), as shown in FIG. 13. The emission scan occurs immediately after the administration of the radionuclide.

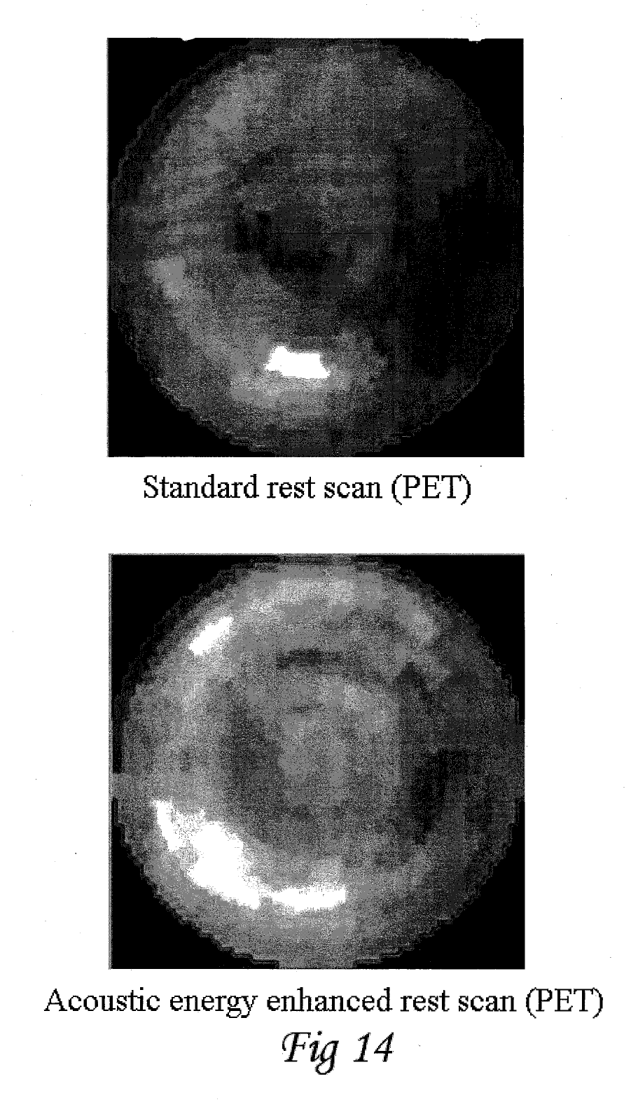

FIG. 14 shows the distribution of the perfusion tracer (rubidium-82) in PET images a slice by slice. A bright orange represents a high blood flow rate, and darker colors, green and blue, represent a low blood flow rate. A mismatch between the pre and post ultrasound image slices with bright shades of orange color show the enhancement of the imaging agent perfusion (rubidium-82), as highlighted in FIG. 14 by white arrows. Enhanced perfusion due to ultrasound treatment means that cells must be viable to take up the perfusion tracer. Patients with viable myocardium are candidates for revascularization.

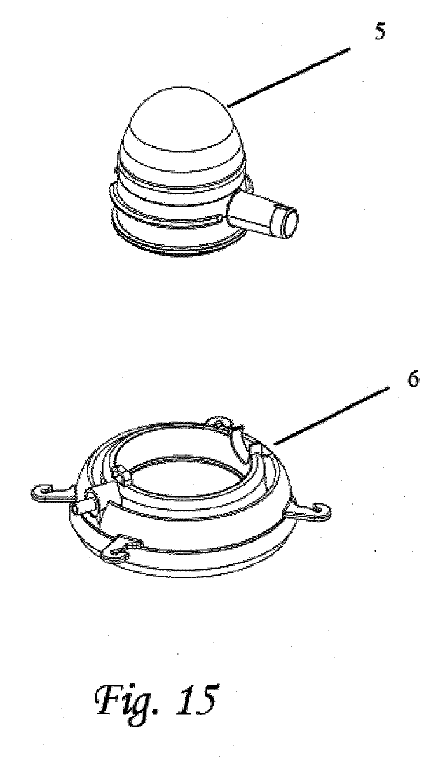

FIG. 15 shows an alternate representation of the images shown in FIG. 14 in a polar map. In a. polar map presentation all individual image slices are combined to one image. The image on left shows the distribution of the perfusion tracer prior to ultrasound treatment, and the image on right shows the same post ultrasound treatment. There is a mismatch in the center of the image and in a sector between 2 o'clock and 4 o'clock. This would suggest that the apex and the left coronary artery region of the heart are “hibernating”, and are viable, and therefore the patient should benefit from revascularization.

The gold standard for myocardial viability testing is PET imaging with FDG (18-fluorine fluorodeoxyglucose). FDG is a metabolism tracer and its distribution in the heart is compared to the distribution of a perfusion tracer, such as 82-rubidium, in PET images. A FDG-to-⁸²Rb mismatch, similar to the one shown in FIG. 15, represents viable myocardium. However, the use of the subject invention instead of FDG PET has several advantages. First, ultrasound enhanced perfusion imaging is faster and easier to implement than FDG PET. Secondly, ultrasound enhanced perfusion imaging reduces exposure to ionizing radiation, and is more comfortable to the patient than PET FDG. In addition, patients with diabetics or low metabolic heart regions such as LBBB (left bundle branch block) who have a limited eligibility to PET FDG viability testing can be tested for myocardial viability with ultrasound enhanced perfusion imaging without the problems associated with PET FDG testing (e.g., blood sugar level monitoring, incomplete data due to low metabolism).

A. The Ultrasound Generator

Returning to the details of the system, FIG. 1 shows a representative embodiment of the ultrasound energy generating machine 16. The machine 16 can also be called an “ultrasound generator.” The machine 16 is intended to be a durable item capable of long term, maintenance free use.

As shown in FIG. 1, the machine 16 can be variously sized and shaped to present a lightweight and portable unit, presenting a compact footprint suited for transport. The machine 16 can be sized and shaped to be mounted at bedside, or to be placed on a table top or otherwise occupy a relatively small surface area. This allows the machine 16 to travel with the patient within an ambulance, airplane, helicopter, or other transport vehicle where space is at a premium. This also makes possible the placement of the machine 16 in a non-obtrusive way within a private home setting.

In the illustrated embodiment, the machine 16 includes a chassis 22, which, for example, can be made of molded plastic or metal or both. The chassis 22 houses a module 24 for generating electric signals. The signals are conveyed to the applicator 18 by an interconnect 30 to be transformed into ultrasonic energy. A controller 26, also housed within the chassis 22 (but which could be external of the chassis 22, if desired), is coupled to the module 24 to govern the operation of the module 24. Further desirable technical features of the controller 26 will be described later.

The machine 16 also preferably includes an operator interface 28. Using the interface 28, the operator inputs information to the controller 26 to affect the operating mode of the module 24. Through the interface 28, the controller 26 also outputs status information for viewing by the operator. The interface 28 can provide a visual readout, printer output, or an electronic copy of selected information regarding the treatment. The interface 28 is shown as being carried on the chassis 22, but it could be located external of the chassis 22 as well.

The machine 16 includes a power cord 14 for coupling to a conventional electrical outlet, to provide operating power to the machine 16. The machine 16 can also include a battery module (not shown) housed within the chassis 22, which enables use of the machine 16 in the absence or interruption of electrical service. The battery module can comprise rechargeable batteries, that can be built in the chassis 22 or, alternatively, be removed from the chassis 22 for recharge. Likewise, the battery module (or the machine 16 itself) can include a built-in or removable battery recharger. Alternatively, the battery module can comprise disposable batteries, which can be removed for replacement.

Power for the machine 16 can also be supplied by an external battery and/or line power module outside the chassis 22. The battery and/or line power module is releasably coupled at time of use to the components within the chassis 22, e.g., via a power distribution module within the chassis 22.

The provision of battery power for the machine 16 frees the machine 16 from the confines surrounding use of conventional ultrasound equipment, caused by their dependency upon electrical service. This feature makes it possible for the machine 16 to provide a treatment modality that continuously “follows the patient,” as the patient is being transported inside a patient transport vehicle, or as the patient is being shuttled between different locations within a treatment facility, e.g., from the emergency room to a holding area within or outside the emergency room.

In a representative embodiment, the chassis 22 measures about 12 inches×about 8 inches×about 8 inches and weighs about 9 pounds.

B. The Ultrasound Applicator

As shown in FIG. 5, the applicator 18 can also be called the “patient interface.” The applicator 18 comprises the link between the machine 16 and the treatment site within the thoracic cavity of the person undergoing treatment. The applicator 18 converts electrical signals from the machine 16 to ultrasonic energy, and further directs the ultrasonic energy to the targeted treatment site.

Desirably, the applicator 18 is intended to be a wholly, or partially disposable item. At least one applicator 18 is coupled to the machine 16 via the interconnect 30 at the beginning a treatment session. The applicator 18 is preferably decoupled from the interconnect 30 (as FIG. 1 shows) and wholly, or partially discarded upon the completing the ultrasound application. However, if desired, the applicator 18 can be designed to accommodate more than a single use.

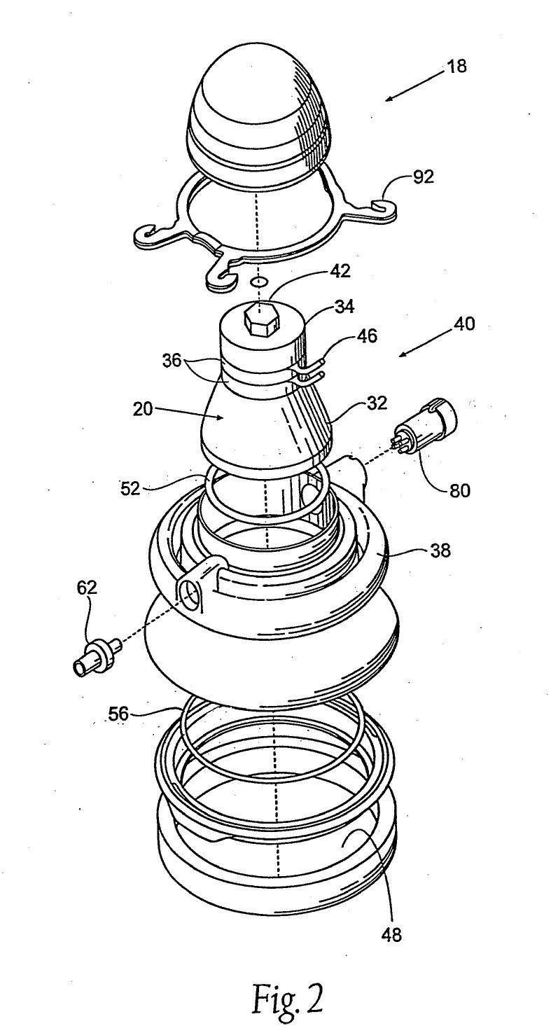

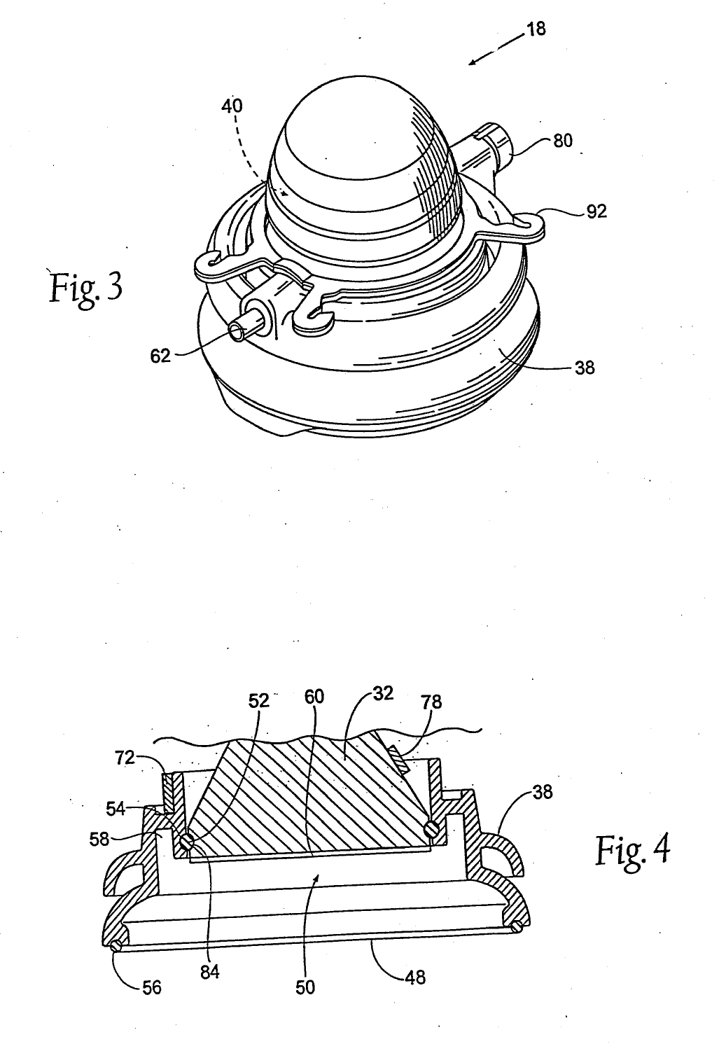

As FIGS. 2 and 3 show, the ultrasound energy applicator 18 includes a shaped metal or plastic body 38 ergonomically sized to be comfortably grasped and manipulated in one hand. The body 38 houses and supports at least one ultrasound transducer 40 (see FIG. 3).

In the illustrated embodiment, the ultrasound transducer 40 comprises an acoustic stack 20. The acoustic stack 20 comprises a front mass piece 32, a back mass piece 34, and one or more piezoelectric elements 36, which are bolted together. The back mass piece 34 comprises an annular ring of material having relatively high acoustic impedance, e.g., steel or stainless steel. “Acoustic impedance” is defined as the product of the density of the material and the speed of sound in the material.

The front mass piece 32 comprises a cone-shaped piece of material having relatively low acoustic impedance, e.g., aluminum or magnesium. The piezoelectric elements 36 are annular rings made of piezoelectric material, e.g., PZT. An internally threaded hole or the like receives a bolt 42 that mechanically biases the acoustic stack 20. A bolt 42 that can be used for this purpose is shown in U.S. Pat. No. 2,930,912. The bolt 42 can extend entirely through the front mass piece 32 or, the bolt 42 can extend through only a portion of the front mass piece 32 (see FIG. 7).

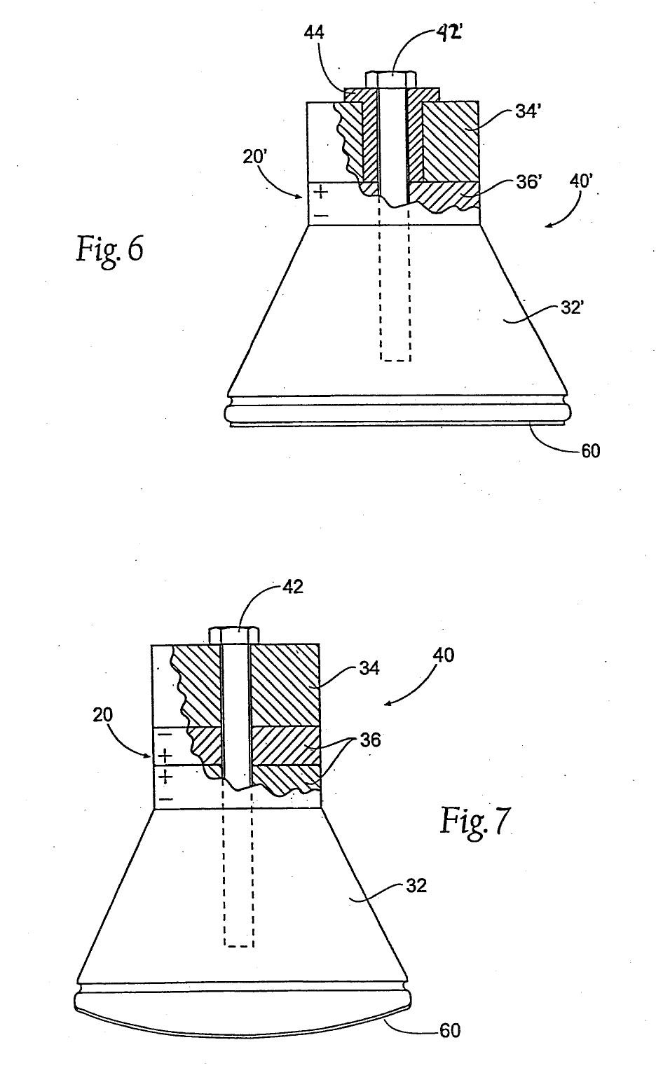

In an alternative embodiment (see FIG. 6), the acoustic stack 20′ of a transducer 40′ can comprise a single piezoelectric element 36′ sandwiched between front and back mass pieces 32′ and 34′. In this arrangement, the back mass piece 34′ is electrically insulated from the front mass piece 32′ by, e.g., an insulating sleeve and washer 44.

The piezoelectric element(s) 36/36′ have electrodes 46 (see FIG. 2) on major positive and negative flat surfaces. The electrodes 46 electrically connect the accoustic stack 20 of the transducer 40 to the electrical signal generating module 24 of the machine 16. When electrical energy at an appropriate frequency is applied to the electrodes 46, the piezoelectric elements 36/36′ convert the electrical energy into mechanical (i.e., ultrasonic) energy in the form of mechanical vibration.

The mechanical vibration created by the transducer 40/40′ is coupled to a patient through a transducer bladder 48, which rests on a skin surface. The bladder 48 defines a bladder chamber 50 (see FIG. 4) between it and the front mass piece 32. The bladder chamber 50 spaces the front mass piece 32 a set distance from the patient's skin. The bladder chamber 50 accommodates a volume of an acoustic coupling media liquid, e.g., liquid, gel, oil, or polymer that is conductive to ultrasonic energy, to further cushion the contact between the applicator 18 and the skin. The presence of the acoustic coupling media also makes the acoustic contact area of the bladder 48 more conforming to the local skin topography.

Desirably, an acoustic coupling medium is also applied between the bladder 48 and the skin surface. The coupling medium can comprise, e.g., a gel material (such as AQUASONIC® 100, by Parker Laboratories, Inc., Fairfield, N.J.). The external material can possess sticky or tacky properties, to further enhance the securement of the applicator 18 to the skin.

In the illustrated embodiment, the bladder 48 and bladder chamber 50 together form an integrated part of the applicator 18. Alternatively, the bladder 48 and bladder chamber 50 can be formed by a separate molded component, e.g., a gel or liquid filled pad, which is supplied separately. A molded gel filled pad adaptable to this purpose is the AQUAFLEX® Ultrasound Gel Pad sold by Parker Laboratories (Fairfield, N.J.).

In a representative embodiment, the front mass piece 32 of the acoustic stack 20 measures about 2 inches in diameter, whereas the acoustic contact area formed by the bladder 48 measures about 4 inches in diameter. An applicator 18 that presents an acoustic contact area of larger diameter than the front mass piece 32 of the transducer 40 makes possible an ergonomic geometry that enables single-handed manipulation during set-up, even in confined quarters, and further provides (with the assembly 12) hands-free stability during use. In a representative embodiment, the applicator 18 measures about 4 inches in diameter about the bladder 48, about 4 inches in height, and weighs about one pound.

An O-ring 52 (see FIG. 4) is captured within a groove 54 in the body 38 of the applicator 18 and a groove 84 on the front mass piece 32 of the transducer 40. The O-ring 52 seals the bladder chamber 50 and prevents liquid in the chamber 50 from contacting the sides of the front mass piece 32. Thus, as FIG. 4 shows, only the outer surface of the front mass piece 32 is in contact with the acoustic coupling medium within the chamber 50.

Desirably, the material of the O-ring 52 is selected to possess elasticity sufficient to allow the acoustic stack 20 of the transducer 40 to vibrate freely in a piston-like fashion within the transducer body 38. Still, the material of the O-ring 52 is selected to be sturdy enough to prevent the acoustic stack 20, while vibrating, from popping out of the grooves 54 and 84.

In a representative embodiment, the O-ring 52 is formed from nitrile rubber (Buna-N) having a hardness of about 30 Shore A to about 100 Shore A. Preferably, the O-ring 52 has a hardness of about 65 Shore A to about 75 Shore A.

The bladder 48 is stretched across the face of the bladder chamber 50 and is preferably also locked in place with another O-ring 56 (see FIG. 4). A membrane ring may also be used to prevent the O-ring 56 from popping loose. The membrane ring desirably has a layer or layers of soft material (e.g., foam) for contacting the skin.

Localized skin surface heating effects may arise by the presence of air bubbles trapped between the acoustic contact area (i.e., the surface of the bladder 48) and the individual's skin. In the presence of ultrasonic energy, the air bubbles vibrate, and thereby may cause cavitation and attendant conductive heating effects at the skin surface. To minimize the collection of air bubbles along the acoustic contact area, the bladder 48 desirably presents a flexible, essentially flat radiating surface contour where it contacts the individual's skin (see FIG. 4), or a flexible, outwardly bowed or convex radiating surface contour (i.e., curved away from the front mass piece) where it contacts with or conducts acoustic energy to the individual's skin. Either a flexible flat or convex surface contour can “mold” evenly to the individual's skin topography, to thereby mediate against the collection and concentration of air bubbles in the contact area where skin contact occurs.

To further mediate against cavitation-caused localized skin surface heating, the interior of the bladder chamber 50 can include a recessed well region 58 surrounding the front mass piece 32. The well region 58 is located at a higher gravity position than the plane of the front mass piece 32. Air bubbles that may form in fluid located in the bladder chamber 50 are led by gravity to collect in the well region 58 away from the ultrasonic energy beam path.

The front mass piece 32 desirably possesses either a flat radiating surface (as FIG. 4 shows) or a convex radiating surface (as FIG. 7 shows). The convex radiation surface directs air bubbles off the radiating surface. The radiating surface of the front mass piece may also be coated with a hydrophilic material 60 (see FIG. 4) to prevent air bubbles from sticking.

The transducer 40 may also include a reflux valve/liquid inlet port 62.

The interconnect 30 carries a distal connector 80 (see FIG. 2), designed to easily plug into a mating outlet in the applicator 18. A proximal connector 82 on the interconnect 30 likewise easily plugs into a mating outlet on the chassis 22 (see FIG. 1), which is itself coupled to the controller 26. In this way, the applicator 18 can be quickly connected to the machine 16 at time of use, and likewise quickly disconnected for discard once the treatment session is over. Other quick-connect coupling mechanisms can be used. It should also be appreciated that the interconnect 30 can be hard wired as an integrated component to the applicator 18 with a proximal quick-connector to plug into the chassis 22, or, vice versa, the interconnect 30 can be hard wired as an integrated component to the chassis 22 with a distal quick-connector to plug into the applicator 18.

As FIG. 5 shows, the stabilization assembly 12 allows the operator to temporarily but securely mount the applicator 18 against an exterior skin surface for use. In the illustrated embodiment, since the treatment site exists in the thoracic cavity, the attachment assembly 54 is fashioned to secure the applicator 18 on the person's thorax, overlaying the sternum or breastbone, as FIG. 5 shows.

The assembly 12 can be variously constructed. As shown in FIG. 5, the assembly 12 comprises straps 90 that pass through brackets 92 carried by the applicator 18. The straps 90 encircle the patient's neck and abdomen.

Just as the applicator 18 can be quickly coupled to the machine 16 at time of use, the stabilization assembly 12 also preferably makes the task of securing and removing the applicator 18 on the patient simple and intuitive. Thus, the stabilization assembly 12 makes it possible to secure the applicator 18 quickly and accurately in position on the patient in cramped quarters or while the person (and the system 10 itself) is in transit.

### III. Controlling the Application of Ultrasound Energy

The system 10 applies ultrasound energy to achieve vasodilation and/or an increase tissue perfusion without causing substantial deep tissue heating. To achieve the optimal application of ultrasound energy and this optimal effect, the system 10 incorporates selection and tuning of an output frequency. The system 10 can also incorporate other features such as power ramping, output power control, and the application of ultrasonic energy at the selected frequency in pulses.

A. Selection of Output Frequency

Depending upon the treatment parameters and outcome desired, the controller 26 desirably operates a given transducer 40 at a fundamental frequency in the range of about 20 kHz or greater. Desirably, the fundamental frequencies lay in a frequency range of about 20 kHz to 500 kHz.

The applicator 18 can include multiple transducers 40 (or multiple applicators 18 can be employed simultaneously for the same effect), which can be individually conditioned by the controller 26 for operation. One or more transducers 40 within an array of transducers 40 can be operated, e.g., at different fundamental frequencies. For example, one or more transducers 40 can be operated at about 20 kHz, while another one or more transducers 40 can be operated at about 50 kHz. More than two different fundamental frequencies can be used, e.g., about 20 kHz, about 50 kHz, and about 100 kHz.

The controller 26 can trigger the fundamental frequency output according to time or a physiological event (such as ECG or respiration).

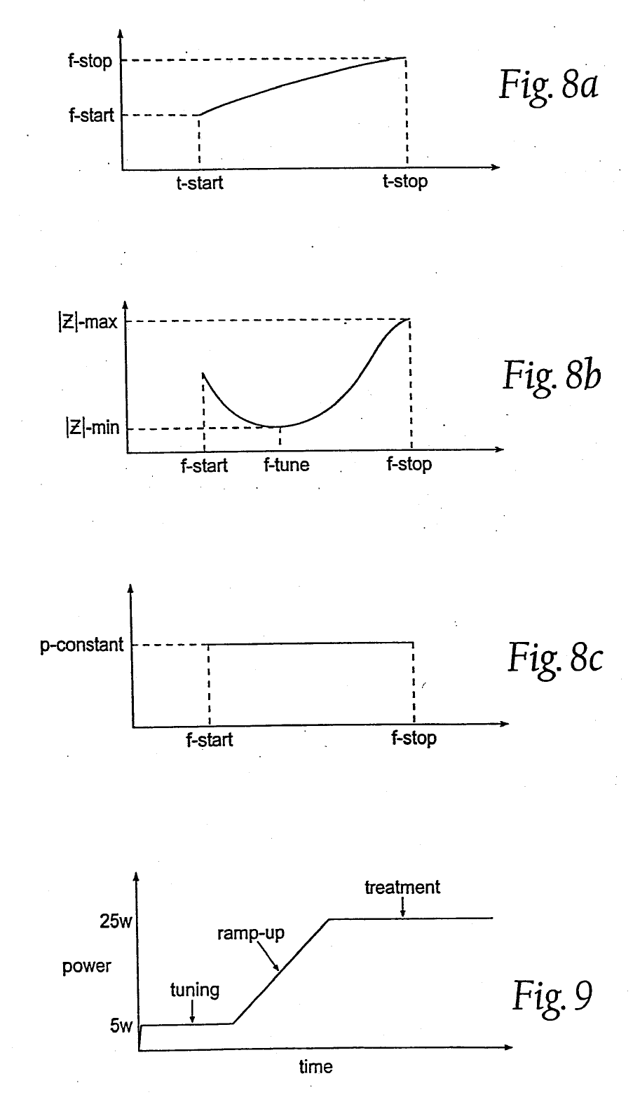

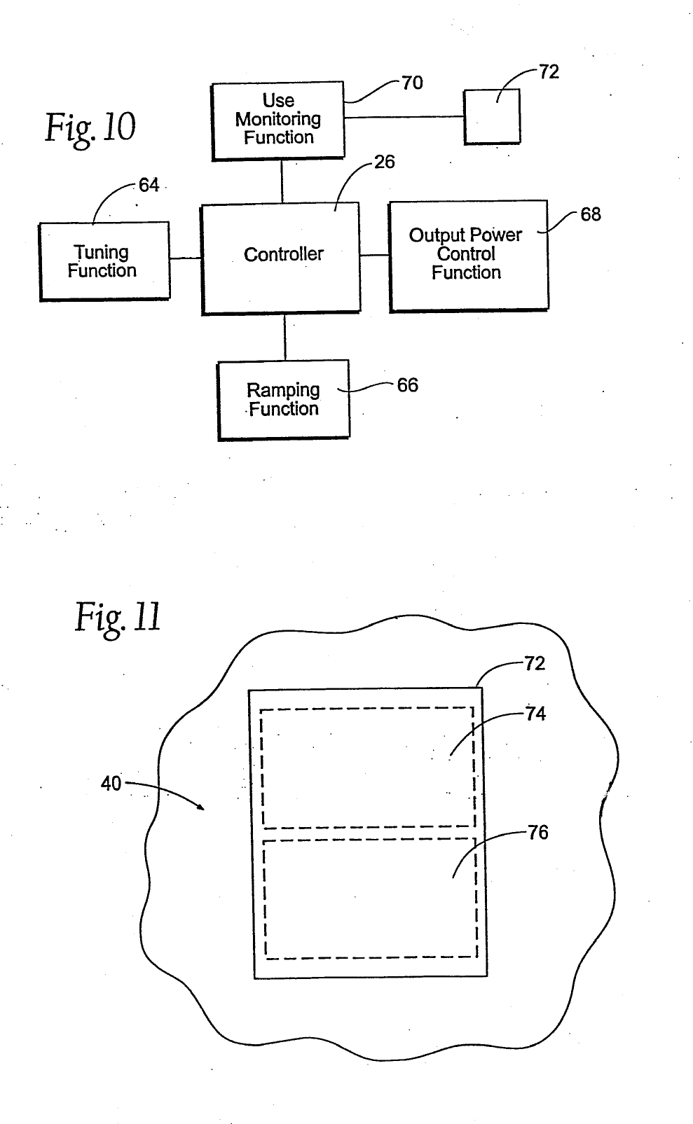

As FIG. 10 shows, the controller 26 desirably includes a tuning function 64. The tuning function 64 selects an optimal frequency at the outset of each treatment session. In the illustrated embodiment (see FIGS. 8A to 8C), the tuning function sweeps the output frequency within a predetermined range of frequencies (f-start to f-stop). The frequency sweep can be and desirably is done at an output power level that is lower than the output power level of treatment (see FIG. 9). The frequency sweep can also be done in either a pulsed or a continuous mode, or in a hybrid mode. An optimal frequency of operation is selected based upon one or more parameters sensed during the sweeping operation.

As FIG. 8A shows, the frequency sweep can progress from a lower frequency (f-start) to a higher frequency (f-stop), or vice versa. The sweep can proceed on a linear basis (as FIG. 8A also shows), or it can proceed on a nonlinear basis, e.g., logarithmically or exponentially or based upon another mathematical function. The range of the actual frequency sweep may be different from the range that is used to determine the frequency of operation. For instance, the frequency span used for the determination of the frequency of operation may be smaller than the range of the actual sweep range.

In one frequency selection approach (see FIGS. 8A and 8C), while sweeping frequencies, the tuning function 64 adjusts the output voltage and/or current to maintain a constant output power level (p-constant). The function 64 also senses changes in transducer impedance (see FIG. 8B)—Z-min to Z-max—throughout the frequency sweep. In this approach (see FIG. 8B), the tuning function 64 selects as the frequency of operation the frequency (f-tune) where, during the sweep, the minimum magnitude of transducer impedance (Z-min) is sensed. Typically, this is about the same as the frequency of maximum output current (I), which in turn, is about the same as the frequency of minimum output voltage (V).

In an alternative frequency selection approach, the tuning function 64 can select as the frequency of operation the frequency where, during the sweep, the maximum of real transducer impedance (Z) occurs, where:

|Z|=√(R²+X²)
and where |Z| is the absolute value of the transducer impedance (Z), which is derived according to the following expression:

Z=R+iX
where R is the real part, and X is the imaginary part.

In another alternative frequency selection approach, while sweeping the frequencies, the tuning function 64 can maintain a constant output voltage. In this approach, the tuning function 64 can select as the frequency of operation the frequency where, during the sweep, the maximum output power occurs. Alternatively, the tuning function 64 can select as the frequency of operation the frequency where, during the sweep, the maximum output current occurs.

B. Power Ramping

As before described, the tuning function 64 desirably operates at an output power level lower than the power level of the actual ultrasound output. In this arrangement, once the operating frequency has been selected, the output power level needs to be increased to the predetermined output level to have the desired effect.

In the illustrated embodiment (see FIG. 10), the controller desirably includes a ramping function 66. The ramping function 66 (see FIG. 9) causes a gradual ramp up of the output power level at which the tuning function 64 is conducted (e.g., 5 W) to the power level at which output occurs (e.g., 25 W). The gradual ramp decreases the possibility of unwanted patient reaction to the ultrasound exposure. Further, a gradual ramp up is likely to be more comfortable to the patient than a sudden onset of the full output power.

In a desired embodiment, the ramping function 66 increases power at a rate of about 0.01 W/s to about 10 W/s. A particularly desired ramping rate is between about 0.1 W/s to about 5 W/s. The ramping function 66 desirably causes the ramp up in a linear fashion (as FIG. 9 shows). However, the ramping function can employ non-linear ramping schemes, e.g., logarithmic, or according to another mathematical function.

C. Output Power Control

Also depending upon the output parameters and outcome desired, the controller 26 can operate a given transducer 40 at a prescribed power level, which can remain fixed or can be varied during the ultrasound output. The controller 26 can also operate one or more transducers 40 within an array of transducers 40 (or when using multiple applicators 18) at a different power levels, which can remain fixed or themselves vary over time.

The parameters affecting power output take into account the output of the signal generator module; the physical dimensions and construction of the applicator; and the physiology of the tissue region to which ultrasonic energy is being applied.

More particularly, the parameters affecting power output can take into account the output of the signal generator module 24; the physical dimensions and construction of the applicator 18; and the physiology of the tissue region to which ultrasonic energy is being applied. In the context of the illustrated embodiment these parameters include the total output power (P_{Total} expressed in Watts [W]) provided to the transducer 40 by the signal generator module 24; the intensity (I, expressed in Watts per square centimeter [W/cm²]) in the effective radiating area of the applicator, which takes into account the total power P_{Total} and the area of the bladder 48; and the peak rarefactional pressure (pᵣ, expressed in Pascals [Pa]) that the tissue experiences.

During the ultrasound output, the transducer impedance may vary due to a number of reasons, e.g., transducer heating, changes in acoustic coupling between the transducer and patient, and/or changes in transducer bladder fill volume, for instance, due to degassing. In the illustrated embodiment (see FIG. 10), the controller 26 includes an output power control function 68. The output power control function 68 holds the output power constant, despite changes in transducer impedance within a predetermined range. If the transducer falls out of the predetermined range, for instance, due to an open or short circuit, the controller 26 shuts down the generator ultrasound energy module 24 and desirably sounds an alarm.

Governed by the output power control function 68, as the transducer impedance increases the output voltage is increased to hold the power output constant. Should the output voltage reach its maximum allowable value, the output power will decrease provided the transducer impedance remains within its predetermined range. As the transducer impedance subsequently drops, the output power will recover and the full output power level will be reached again.

Governed by the output power control function 68, as the transducer impedance decreases, the output current is increased to hold the power output constant. Should the output current reach a preset maximum allowable value, the output power will decrease until the impedance increases again, and will allow full output power.

In addition to the described changes in the output voltage and current to maintain a constant output power level, the output power control function 68 can vary the frequency of operation slightly upward or downward to maintain the full output power level within the allowable current and voltage limits.

D. Pulsed Power Mode

The application of ultrasound energy in a pulsed power mode serves, in conjunction with the selection of the fundamental output frequency, to reduce deep heating tissue effects. This is because, at a given frequency, a high ultrasound intensity, or high ultrasound power, results in more tissue heating than a low intensity, or power. At the same peak ultrasound intensity, the pulse mode application of ultrasound results in less tissue heating than continuous mode because heat is dissipated in between the pulses. During the pulsed power mode, ultrasound is applied at a desired fundamental frequency or within a desired range of fundamental frequencies at the prescribed power level or range of power levels (as described above, to achieve desired physiological effect) in a prescribed duty cycle (DC) or range of duty cycles and a prescribed pulse repetition frequency (PRF) or range of pulse repetition frequencies. Desirably, the pulse repletion frequency (PRF) is between about 20 Hz to about 50 Hz (i.e., between about 20 pulses a second and about 50 pulses a second).

The duty cycle (DC) is equal to the pulse duration divided by one over the pulse repetition frequency (PRF). The pulse repletion frequency (PRF) represents the amount of time from the beginning one pulse to the beginning of the next pulse. For example, given a pulse repletion frequency (PRF) of 30 Hz (30 pulses per second) and a duty cycle of 25% yields a pulse duration of approximately 8 ms pulse followed by a 25 ms off period 30 times per second.

Given a pulse repetition frequency (PRF) selected at 25 Hz and a desired fundamental frequency between about 20 kHz and 500 kHz delivered in a power range between about 5 to 30 W, a duty cycle of about 50% or less meets the desired physiological objectives with less incidence of localized conductive heating effects compared to a continuous application of the same fundamental frequency and power levels over a comparable period of time. Given these operating conditions, the duty cycle desirably lays in a range of between about 1% and about 35%.

### IV. Monitoring Use of the Transducer

To protect patients from the potential adverse consequences occasioned by multiple use, which include disease transmission, or material stress and instability, or decreased or unpredictable performance, the controller 26 desirably includes a use monitoring function 70 (see FIG. 10) that monitors incidence of use of a given transducer 40.

In the illustrated embodiment, the transducer 40 carries a use register 72 (see FIG. 4). The, use register 72 is configured to record information before, during, and after a given treatment session. The use register 72 can comprise a solid state micro-chip, ROM, EBROM, EPROM, or non volatile RAM (NVRAM) carried by the transducer 40.

The use register 72 is initially formatted and programmed by the manufacturer of the system to include memory fields. In the illustrated embodiment (see FIG. 11), the memory fields of the use register are of two general types: Write Many Memory Fields 74 and Write-Once Memory Fields 76. The Write Many Memory Fields 74 record information that can be changed during use of the transducer 40. The Write-Once Memory Fields 76 record information that, once recorded, cannot be altered.

The specific information recorded by the Memory Fields 74 and 76 can vary. The following table exemplifies typical types of information that can be recorded in the Write Many Memory Fields 74.

| | | Size |
|---|---|---|
| Field Name | Description | (Byte) |
| Treatment | If a transducer has been used for a prescribed | 1 |
| Complete | maximum treatment time (e.g., 60 minutes), the | |
| | treatment complete flag is set to 1 otherwise it is | |
| | zero. | |
| Prescribed | This is the allowable usage time of the transducer. | 2 |
| Maximum | This is set by the manufacturer and determines at | |
| Treatment Time | what point the Treatment Complete flag is set to 1. | |
| (Minutes) | | |
| Elapsed Usage | Initialized to zero. This area is then incremented | 2 |
| Time (Minutes) | every minute that the system is transmitting acoustic | |
| | energy. This area keeps track of the amount of time | |
| | that the transducer has been used. When this time | |
| | reaches the Prescribed Maximum Treatment Time, | |
| | the treatment Complete flag is set to 1. | |
| Transducer | This is an area that could be used to prescribe the | 2 |
| Frequency | operational frequency of the transducer, rather than | |
| | tuning the transducer to an optimal frequency, as | |
| | above described. In the latter instance, this area | |
| | shows the tuned frequency once the transducer has | |
| | been tuned. | |
| Average Power | The system reads and accumulates the delivered | 2 |
| | power through the procedure. Every minute, the | |
| | average power number is updated in this area from | |
| | the system, at the same time the Elapses Usage | |
| | Time is updated. This means that the average | |
| | power reading could be off by a maximum of 59 | |
| | seconds if the treatment is stopped before the | |
| | Treatment Complete flag is set. This average power | |
| | can be used as a check to make sure that the | |
| | system was running at full power during the | |
| | procedure. | |
| Applicator | Use Register CRC. This desirably uses the same | 2 |
| CRC | CRC algorithm used to protect the controller ROM | |
| Copyright Notice | Desirably, the name of the manufacturer is recorded | 11 |
| | in this area. Other information can be recorded here | |
| | as well. | |

The on/off cycles of ultrasound energy transmission could affect the accuracy of the recorded power levels because of the variance of the power levels due to ramping function 66. For this reason it may be advantageous to also record the number of on/off cycles of ultrasound energy transmission. This will help explain any discrepancies in average power reading. It might also allow the identification of procedural problems with system use.

Each use register 72 can be assigned a unique serial number that could be used to track transducers in the field. This number can be read by the use monitoring function 70 if desired.

The following table exemplifies typical types of information that can be recorded in the Write-Once Memory Fields 76.

| | | | Size |
|---|---|---|---|
| Field Name | Description | Location | (Byte) |
| Start Date | One the system has tuned the | | |
| Time | transducer and started to transmit | | |
| | ultrasound energy, the current date | | |
| | and time are written to this area. | | |
| | This area is then locked, which | | |
| | prevents the data from ever being | | |
| | changed. | | |
| Tuned | The tuned frequency is written to | | |
| Frequency | this location when the Start Date | | |
| | and Time is set. This prevents this | | |
| | information from being written over | | |
| | on subsequent tunes (if necessary). | | |

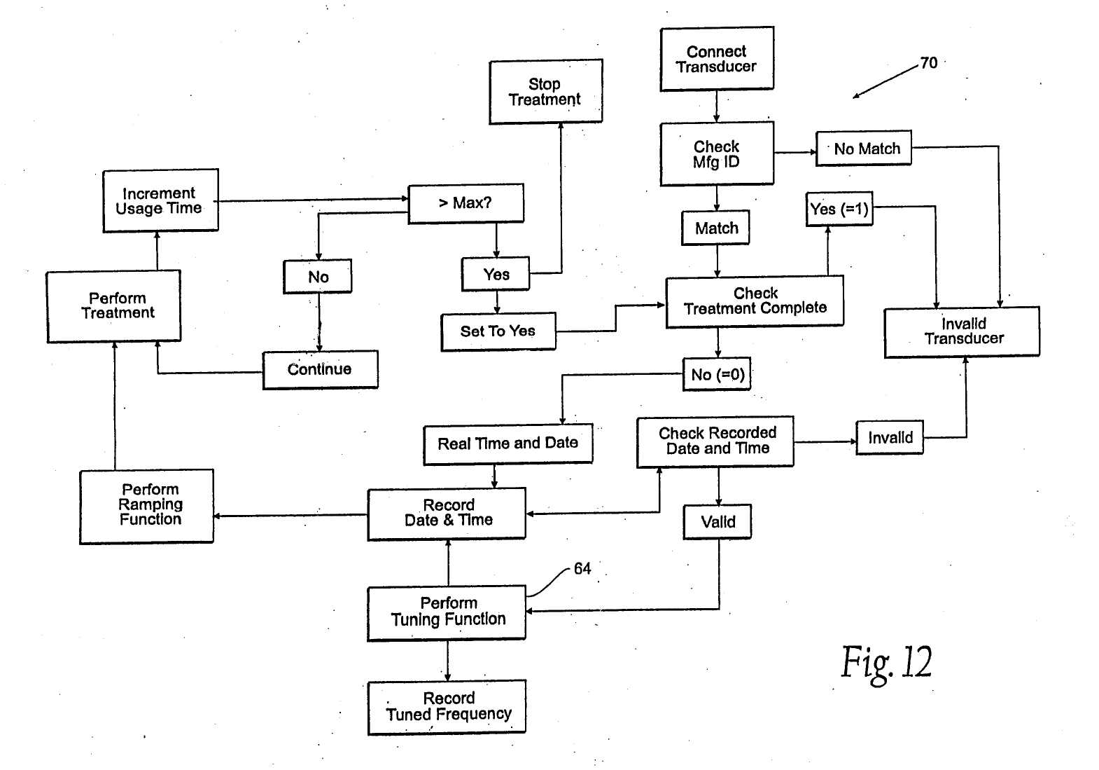

As FIG. 12 shows, when a transducer 40 is first coupled to the machine 16, and prior to enabling the conveyance of ultrasound energy to the transducer 40, the use monitoring function 70 prompts the use register 72 to output resident information recorded in the memory fields.

The use monitoring function 70 compares the contents of the Copyright Notice field to a prescribed content. In the illustrated embodiment, the prescribed content includes information contained in the Copyright Notice field of the Write Many Memory Fields 74. The prescribed content therefore includes the name of the manufacturer, or other indicia uniquely associated with the manufacture. If the prescribed content is missing, the use monitoring function 70 does not enable use of the transducer 40, regardless of the contents of any other memory field. The transducer 40 is deemed “invalid.” In this way, a manufacturer can assure that only transducers meeting its design and quality control standards are operated in association with the machine 16.

If the contents of the Copyright Notice field match, the use monitoring function 70 compares the digital value residing in the Treatment Complete field of the Write Many Memory Fields 74 to a set value that corresponds to a period of no prior use or a prior use less than the Prescribed Maximum Treatment Time i.e., in the illustrated embodiment, a zero value. A different value (i.e., a 1 value) in this field indicates a period of prior use equal to or greater than the Prescribed Maximum Treatment Time. In this event, the use monitoring function 70 does not enable-use of the transducer 40. The transducer 40 is deemed “invalid.”

If a value of zero resides in the Treatment Complete field, the use monitoring function 70 compares the date and time data residing in the Write-Once Start Date and Time field to the current date and time established by a Real Time Clock. If the Start Date and Time is more than a prescribed time before the Real Time (e.g., 4 hours), the controller does not enable use of the transducer 40. The transducer 40 is deemed “invalid.”

If the Start Date and Time field is empty, or if it is less than the prescribed time before the Real Time, the use monitoring function 70 deems the transducer 40 to be “valid”(providing the preceding other criteria have been met). The use monitoring function 70 reports a valid transducer to the controller 26, which initiates the tuning function 64. If the Start Date and Time field is empty, once the tuning function 64 is completed, the controller prompts the use monitoring function 70 to record the current date and time in the Start Date and Time Field, as well as the selected operating frequency in the Tuned Frequency field. The controller 26 then proceeds to execute the ramping function 66 and, then, executes the prescribed treatment protocol

If the Start Date and Time field is not empty (indicating a permitted prior use), once the tuning function 64 is completed; the controller 26 immediately proceeds with the ramping function 66 and, then, executes the treatment protocol.

During use of the transducer 40 to accomplish the treatment protocol, the use monitoring function 70 periodically updates the Elapsed Usage Time field and Average Power field (along with other Many Write Memory Fields). Once the Treatment Complete flag is set to a 1 value (indicating use of the transducer beyond the Prescribed Maximum Treatment Time), the use monitoring function 70 interrupts the supply of energy to the transducer. The transducer 40 is deemed “invalid” for subsequent use. The use monitoring function 70 can also generate an output that results in a visual or audible alarm, informing the operator that the transducer 40 cannot be used.

The information recorded in the use register 72 can also be outputted to monitor use and performance of a given transducer 40. Other sensors can be used, e.g., a temperature sensor 78 carried on the front mass piece 32 (see FIG. 4), in association with the use register.

As described, the use register 72 allows specific pieces of information to be recorded before, during and after a treatment is complete. Information contained in the use register 72 is checked before allowing use of a given transducer 40. The use register 72 ensures that only a transducer 40 having the desired design and performance criteria imparted by the manufacturer can be used. In addition, the use register 72 can be used to “lock out” a transducer 40 and prevent it from being used in the future. The only way the transducer 40 could be reused is to replace the use register 72 itself. However, copying the architecture of the use register 72 (including the contents of the Copyright Message field required for validation) itself constitutes a violation of the manufacturer's copyright in a direct and inescapable way.

What is claimed is:

1. A method for cardiac diagnostic imaging comprising the steps of:
delivering acoustic energy to the myocardial tissue,
performing a myocardial imaging procedure on a patient, and
determining myocardial tissue perfusion.

2. The method of claim 1, wherein a nuclear imaging agent is delivered to the patient.

3. The method of claim 2, wherein the nuclear imaging agent is selected from the group consisting of thalium-201, technetium-99m, nitrogen-13 ammonia, rubidium-82, and oxygen-15 water.

4. A method for cardiac diagnostic imaging comprising the steps of:
performing a first myocardial imaging procedure on a patient,
delivering acoustic energy to the myocardial tissue before, during, or after the first myocardial imaging procedure,
performing a second myocardial imaging procedure on a patient, and
determining whether the acoustic energy has changed tissue perfusion.

5. The method of claim 4, wherein at least one myocardial imaging procedure is conducted while the patient is at rest.

6. The method of claim 4, wherein the myocardial imaging procedure is selected from the group of nuclear imaging, perfusion imaging, positron emission imaging, and single photon emission imaging.

7. A method for cardiac diagnostic imaging comprising the steps of:
delivering acoustic energy to the myocardial tissue,
performing a myocardial imaging procedure on a patient, and
determining myocardial blood flow.

8. A method for cardiac diagnostic imaging comprising the steps of:
performing a first myocardial imaging procedure on a patient,
delivering acoustic energy to the myocardial tissue before, during, or after the first myocardial imaging procedure,
performing a second myocardial imaging procedure on a patient, and
determining whether the acoustic energy has changed myocardial blood flow.

9. A method for cardiac diagnostic imaging comprising the steps of:
delivering acoustic energy to the myocardial tissue,
performing a myocardial imaging procedure on a patient, and
determining cardiac motion.

10. A method for cardiac diagnostic imaging comprising the steps of:
performing a first myocardial imaging procedure on a patient,
delivering acoustic energy to the myocardial tissue before, during, or after the first myocardial imaging procedure,
performing a second myocardial imaging procedure on a patient, and
determining whether the acoustic energy has changed cardiac motion.

11. A method for diagnostic imaging comprising the steps of:
delivering acoustic energy to a target tissue,
performing an imaging procedure on a patient, and
determining blood flow to or within the target tissue.

12. In a device for delivering acoustic energy, the improvement comprising:
a chassis,
an applicator for delivering acoustic energy, said applicator having a surface though which acoustic energy is delivered, and wherein said surface is removably connected to the applicator.
